(19) United States
(12) Patent Application Publication
(10) Pub. No.: US 2022/0144768 A1
(43) Pub. Date: May 12, 2022

(54) SOLID STATE FORMS OF SIPONIMOD

(71) Applicant: DR. REDDY'S LABORATORIES LIMITED, Hyderabad (IN)

(72) Inventors: Srividya RAMAKRISHNAN, Hyderabad (IN); Vamsi Krishna MUDAPAKA, Bhadrachalam (IN); Satyanarayana THIRUNAHARI, Hyderabad (IN); Srinivasulu RANGINENI, Ameenpur (IN)

(21) Appl. No.: 17/433,073

(22) PCT Filed: Feb. 26, 2020
(86) PCT No.: PCT/IB2020/051619

§ 371 Date: Aug. 23, 2021

(30) Foreign Application Priority Data
Feb. 27, 2019 (IN) 201941007652
Apr. 17, 2019 (IN) 201941015509

(51) Int. Cl. C07D 205/04

(52) U.S. Cl. CPC C07D 205/04; C07B 2200/13

(57) ABSTRACT

The present application provides novel polymorphic forms of siponimod, their processes, their use in purification of other crystalline polymorphic forms of siponimod, and pharmaceutical compositions containing them. The present application specifically provides crystalline Form S, Form S1 and Form S2 of siponimod, their preparative methods, their use in purification of other crystalline forms of siponimod and pharmaceutical compositions thereof.

### FIELD OF THE INVENTION

The present application relates to crystalline and amorphous solid forms of Siponimod, their preparative methods and pharmaceutical compositions thereof.

### BACKGROUND OF THE INVENTION

The drug compound having the adopted name Siponimod, has a chemical name (E)-1-(4-(1-(((4-cyclohexyl-3-(trifluoromethyl)benzyl)oxy)-imino)ethyl)-2-ethylbenzyl)-azetidine-3-carboxylic acid, and is represented by the structure of formula I.

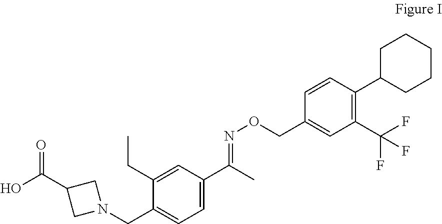 [SMILES: CCc1cc(/C(C)=N/OCc2ccc(C3CCCCC3)c(C(F)(F)F)c2)ccc1CN1CC(C(=O)O)C1]

Siponimod is a selective sphingosine-1-phosphate receptor modulator drug approved in USA for the treatment of secondary progressive multiple sclerosis.

Siponimod, its synthetic process and its pharmaceutical compositions are described in U.S. Pat. No. 7,939,519 B2 (US '519). Siponimod hemifumarate salt and its pharmaceutical compositions are described in US patent application No. 20150175536 A1 (US '536).

The US '536 also describes crystalline forms of Siponimod hemifumarate salt and their pharmaceutical compositions.

International patent application, WO 2019/064184 A1, published on 4 Apr. 2019, describes crystalline solid state forms of Siponimod hemifumarate and siponimod monofumarate.

Polymorphism, the occurrence of different crystal forms, is a phenomenon of some molecules and molecular complexes. A single molecule may give rise to a variety of polymorphs having distinct crystal structures and physical properties. Polymorphs in general will have different melting points, thermal behaviors (e.g. measured by thermogravimetric analysis—“TGA”, or differential scanning calorimetry—“DSC”), X-ray powder diffraction (XRPD or powder XRD) pattern, infrared absorption fingerprint, and solid state nuclear magnetic resonance (NMR) spectrum. One or more of these techniques may be used to distinguish different polymorphic forms of a compound.

Discovering new polymorphic forms, hydrates and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound or salts thereof can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. For at least these reasons, there is a need for additional solid forms of Siponimod.

### SUMMARY OF THE INVENTION

Aspects of the present application relate to novel solid state forms of Siponimod, their preparative processes and pharmaceutical compositions thereof.

In one aspect, the present application provides a crystalline Form S of Siponimod, characterized by a PXRD pattern comprising the peaks at about 6.95, 10.44, 12.12, 12.30, 17.09 and 22.11±0.2° 2θ.

In another aspect, the present application provides a process for the preparation of crystalline Form S of Siponimod, comprising,

(a) providing a mixture of Siponimod fumaric acid co-crystal and glycerin,

(b) adding an alcohol solvent to the mixture of step (a), and

(c) isolating the crystalline Form S of Siponimod.

In another aspect, the present application provides a crystalline Form S1 of Siponimod, characterized by a PXRD pattern comprising the peaks at about 7.18, 10.76, 12.0, 20.08 and 21.62±0.2° 2θ.

In another aspect, the present application provides a process for the preparation of crystalline Form S1 of Siponimod, comprising,

(a) providing a mixture of crystalline Siponimod Form S and water,

(b) stirring the mixture of step (a), and

(c) isolating the crystalline Form S1 of Siponimod.

In another aspect, the present application provides a crystalline Form S2 of Siponimod, characterized by a PXRD pattern comprising the peaks at about 12.03, 17.68 and 20.09±0.2° 2θ.

In another aspect, the present application provides a process for the preparation of crystalline Form S2 of Siponimod, comprising drying the crystalline Form S1 of Siponimod at about 25° C. to about 60° C.

In another aspect, the present application provides a process for preparation of crystalline Form S2 of Siponimod, comprising,

(a) providing a mixture of Siponimod fumaric acid co-crystal, water and a base,

(b) stirring the mixture of step (a),

(c) optionally, adding the mixture with a suitable acid, and

(d) isolating the crystalline Form S2 of Siponimod.

In another aspect, the present application provides a process for the preparation of crystalline Form S2 of Siponimod, comprising,
(a) providing a mixture of crystalline form of Siponimod and water,
(b) stirring the mixture of step (a), and
(c) isolating the crystalline Form S2 of Siponimod.

In one aspect, the present application provides a crystalline Form SMA1 of Siponimod, characterized by a PXRD pattern comprising a peak at 17.85±0.2° 2θ.

In another aspect, the present application provides a process for the preparation of crystalline Form SMA1 of Siponimod, comprising,

(a) providing a mixture of Siponimod, malic acid and a solvent,

(b) stirring the mixture of step (a), and

(c) isolating the crystalline Form SMA1 of Siponimod.

In another aspect, the present application provides a crystalline Form SME1 of Siponimod, characterized by a PXRD pattern comprising the peaks at about 3.64, 15.56, 16.57, 19.44 and 22.00±0.2° 2θ.

In another aspect, the present application provides a process for the preparation of crystalline Form SME1 of Siponimod, comprising,

(a) providing a mixture of Siponimod, maleic acid and a solvent,

(b) stirring the mixture of step (a), and

(c) isolating the crystalline Form SME1 of Siponimod.

In another aspect, the present application provides amorphous form of Siponimod.

In another aspect, the present application provides a process for preparing amorphous form of Siponimod, which comprises;
a) providing a solution of a pharmaceutically acceptable salt or a co-crystal of Siponimod in a solvent;
b) adding a base to the solution obtained in step (a); and
c) isolating amorphous form of Siponimod.

In another aspect, the present application provides a process for preparing amorphous form of Siponimod, comprising,

a) providing a solution of Siponimod in a solvent or a mixture of solvents;

b) removing solvent from the solution of Siponimod obtained in step a); and

c) recovering amorphous form of Siponimod.

In another aspect, the present application provides use of the crystalline forms of Siponimod to improve the purity of Siponimod and its salts.

In another aspect, the present application provides a pharmaceutical composition comprising any of the crystalline forms of Siponimod of the present application and at least one pharmaceutically acceptable carrier.

### BRIEF DESCRIPTION OF THE DRAWINGS

FIG. 1 is powder X-ray diffraction (“PXRD”) pattern of crystalline form S of Siponimod prepared according to Example 1.

FIG. 2 is powder X-ray diffraction pattern of crystalline form S1 of Siponimod prepared according to Example 3.

FIG. 3 is powder X-ray diffraction pattern of crystalline form S2 of Siponimod prepared according to Example 7.

FIG. 4 is powder X-ray diffraction (“PXRD”) pattern of crystalline form SMA1 of Siponimod prepared according to Example 11.

FIG. 5 is powder X-ray diffraction pattern of crystalline form SME1 of Siponimod prepared according to Example 12.

FIG. 6 is powder X-ray diffraction pattern of amorphous form of Siponimod prepared according to Example 13.

FIG. 7 is powder X-ray diffraction pattern of amorphous form of Siponimod prepared according to Example 15.

### DETAILED DESCRIPTION

Aspects of the present application relate to novel crystalline solid forms of Siponimod, their preparative processes and pharmaceutical compositions thereof. The present application also encompasses the use of novel crystalline solid forms of Siponimod provided herein, for the preparation of other solid forms of Siponimod and its salts, for the purification of Siponimod fumaric acid co-crystal and for the preparation of pharmaceutical dosage forms.

In one aspect, the present application provides a crystalline Form S of Siponimod, characterized by a PXRD pattern comprising the peaks at about 6.95, 10.44, 12.12, 12.30, 17.09 and 22.11±0.2° 2θ.

In another aspect, the present application provides a process for the preparation of crystalline Form S of Siponimod, comprising,

(a) providing a mixture of Siponimod fumaric acid co-crystal and glycerin,

(b) adding an alcohol solvent to the mixture of step (a), and

(c) isolating the crystalline Form S of Siponimod.

The step (a) involves addition of Siponimod fumaric acid co-crystal into hot glycerin. Any physical form of Siponimod fumaric acid co-crystal may be used as starting material. Preferably, Siponimod fumaric acid co-crystal is in its crystalline Form A. The Glycerin may be heated to about 60° C. to about 100° C. After adding the Siponimod fumaric acid co-crystal into hot glycerin the mixture may be stirred for about 10 minutes to about 5 hours at about 60° C. to about 100° C. The step (b) involves adding an alcohol solvent to the mixture of step (a). The alcohol solvent is selected form the group comprising methanol, ethanol, isopropyl alcohol, tert-butyl alcohol and amyl alcohol. Preferably, the alcohol solvent is methanol. The resulted mixture may be stirred at 60° C. to get complete dissolution. After complete dissolution the mixture may be filtered to get rid of particulate matter.

The step (c) involves isolation of solid from the mixture. Isolation of the solid may be carried out by any methods known in the art or procedures described in the present application. In an embodiment, the solid is isolated by slow evaporation of the solvent. The isolated solid may be washed with a suitable solvent like methyl tert-butyl ether to obtain the crystalline Form S of Siponimod.

In another aspect, the crystalline Form S of Siponimod is further characterized by a PXRD pattern comprising the peaks at about 13.68, 13.93, 20.54, 23.59, 26.20 and 31.66±0.2° 2θ.

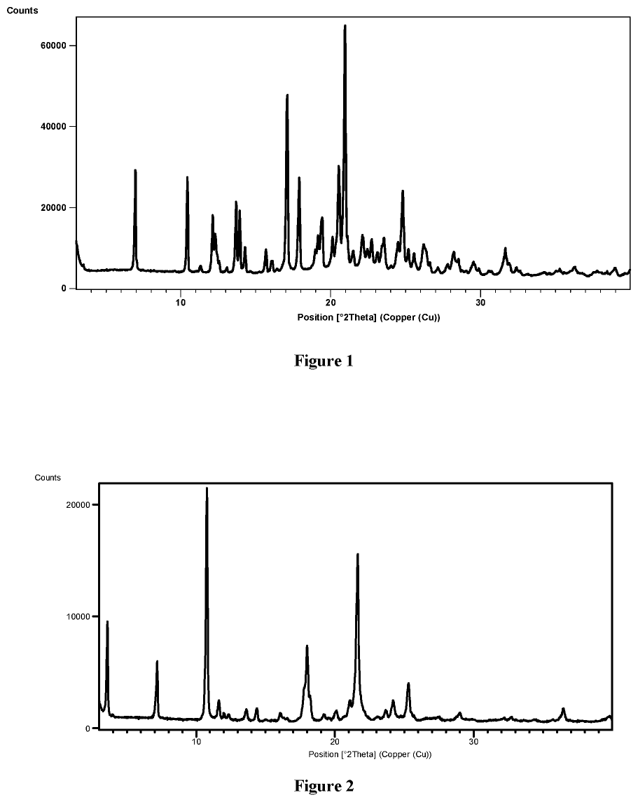

In another aspect, the crystalline Form S of Siponimod is characterized by the PXRD pattern of FIG. 1.

In another aspect, the present application provides a crystalline Form S1 of Siponimod, characterized by a PXRD pattern comprising the peaks at about 7.18, 10.76, 12.0, 20.08 and 21.62±0.2° 2θ.

In another aspect, the present application provides a process for the preparation of crystalline Form S1 of Siponimod, comprising,

(a) providing a mixture of crystalline Form S Siponimod and water,

(b) stirring the mixture of step (a), and

(c) isolating the crystalline Form S1 of Siponimod.

The step (a) involves providing a mixture of Form S of Siponimod and water. The Form S is prepared by the process described in this application. Water may be taken in the ratio of 1:5 to 1:100 w/v with respect to siponimod Form S. The step (b) involves stirring the mixture of step (a) for about 10 minutes to about 10 hours at about 20° C. to about 60° C.

The step (c) involves isolation of solid from the mixture. Isolation of the solid may be carried out by filtering the suspension. The isolated solid is identified as crystalline Form S1 of Siponimod.

In another embodiment, the crystalline Form S1 of Siponimod is further characterized by a PXRD pattern comprising the peaks at about 3.59, 11.62, 13.61 and 14.36±0.2° 2θ.

In another aspect, the crystalline Form S1 of Siponimod is characterized by the PXRD pattern of FIG. 2.

In another aspect, the present application provides a crystalline Form S2 of Siponimod, characterized by a PXRD pattern comprising the peaks at about 12.03, 17.68 and 20.09±0.2° 2θ.

In another aspect, the present application provides a process for the preparation of crystalline Form S2 of Siponimod, comprising drying the crystalline Form S1 of Siponimod at about 25° C. to about 60° C.

The drying may be carried out using an air tray dryer or a vacuum tray dryer. And the drying of the crystalline Form S1 of Siponimod may be done at 25° C. to about 60° C. for a period of about 10 minutes to about 10 hours.

In another aspect, the present application provides a process for preparation of crystalline Form S2 of Siponimod, comprising,

(a) providing a mixture of Siponimod fumaric acid co-crystal, water and a base

(b) stirring the mixture of step (a),

(c) optionally, adding the mixture with a suitable acid, and

(d) isolating the crystalline Form S2 of Siponimod.

The step (a) involves mixing of Siponimod fumaric acid co-crystal, water and a base. Any polymorphic form of Siponimod fumaric acid co-crystal may be used as starting material. Preferably, the polymorphic form A of Siponimod fumaric acid co-crystal, described in US patent application No. 20150175536 A1, is used as starting material. The base may be selected from the group comprising sodium hydroxide, lithium hydroxide, potassium hydroxide, lithium bicarbonate, sodium bicarbonate, potassium bicarbonate, lithium carbonate, sodium carbonate, potassium carbonate, ammonia and the like. Preferably, the base is sodium bicarbonate.

The step (b) involves stirring the mixture of step (a) at about 15° C. to about 45° C. for about 30 minutes to about 30 hours. In step (c) an acid may be added to the mixture optionally. The acid may be selected from the group comprising acetic acid, hydrochloric acid, sulphuric acid. Preferably, the acid is aqueous acetic acid.

The step (d) involves isolation of crystalline Form S2 of Siponimod from the mixture. The isolation may be carried out by filtering the suspension. The isolated solid is dried at about 20° C. to about 40° C. for about 1 hour to about 20 hours to yield crystalline Form S2 of Siponimod.

In another aspect, the present application provides a process for preparation of crystalline Form S2 of Siponimod, comprising,
(a) providing a mixture of crystalline form of Siponimod and water,
(b) stirring the mixture of step (a), and
(c) isolating the crystalline Form S2 of Siponimod.

The step (a) involves mixing of crystalline form of Siponimod and water. Preferably, the crystalline form A of Siponimod described in U.S. Pat. No. 8,173,634 B2, is used as starting material. The step (b) involves stirring the mixture of step (a) at about 15° C. to about 65° C. for about 30 minutes to about 30 hours. The step (c) involves isolation of crystalline Form S2 of Siponimod from the mixture. Isolation may be carried out by filtering the suspension. The isolated solid is dried at about 20° C. to about 40° C. for about 10 minutes to about 10 hours to yield crystalline Form S2 of Siponimod.

In another embodiment, the crystalline Form S2 of Siponimod is further characterized by a PXRD pattern comprising the peaks at about 4.03 and 8.02±0.2° 2θ.

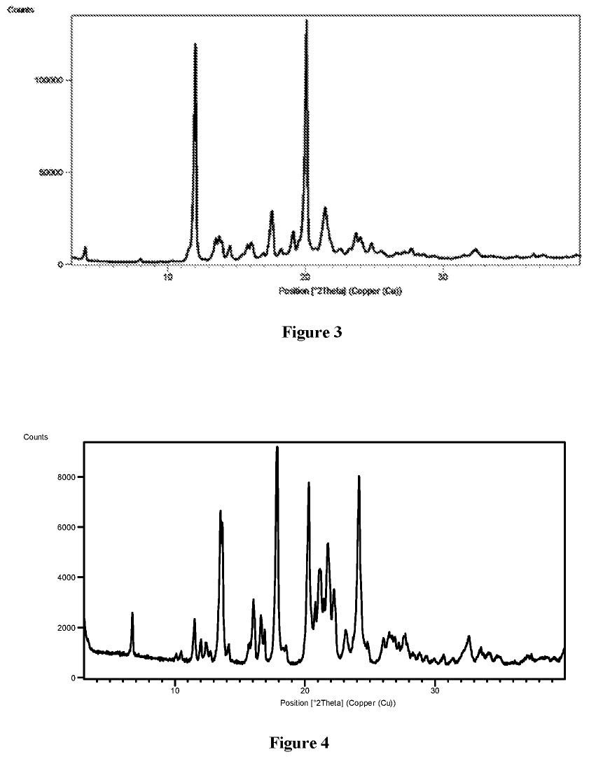

In another aspect, the crystalline Form S2 of Siponimod is characterized by the PXRD pattern of FIG. 3.

The crystalline Form S2 of Siponimod of the present application is stable for at least three months. The crystalline Form S2 of Siponimod prepared by the process of the present application is kept at about 25° C. After three months the solid was analyzed using PXRD. The solid remains in crystalline Form S2 of Siponimod and the PXRD pattern matches with that of FIG. 3. The crystalline Form S2 of Siponimod prepared by the process of the present application is kept at about 70° C. After three months the solid was analyzed using PXRD. The solid remains in crystalline Form S2 of Siponimod and the PXRD pattern matches with that of FIG. 3.

In one aspect, the present application provides a crystalline Form SMA1 of Siponimod, characterized by a PXRD pattern comprising the peak at about 17.85±0.2° 2θ.

In another aspect, the present application provides a process for the preparation of crystalline Form SMA1 of Siponimod, comprising,

(a) providing a mixture of Siponimod, malic acid and a solvent,

(b) stirring the mixture of step (a), and

(c) isolating the crystalline Form SMA1 of Siponimod.

The step (a) involves providing a mixture of Siponimod, malic acid and a suitable solvent. Siponimod may be prepared by the process described in the art or may be prepared by the process described in this application. The malic acid is D,L-malic acid and is used in 1:1 mole ratio with respect to Siponimod. The solvent may be taken in the ratio of 1:10 to 1:50 w/v with respect to Siponimod. The step (b) involves stirring the mixture of step (a) for about an hour to about 50 hours at above 30° C.

The step (c) involves isolation of solid from the mixture. Isolation of the solid may be carried out by any methods known in the art or procedures described in the present application. In an embodiment, the solid is isolated by filtration. The isolated solid may be washed with a suitable solvent to obtain the crystalline Form SMA1 of Siponimod.

In another aspect, the crystalline Form SMA1 of Siponimod is further characterized by a PXRD pattern comprising the peaks at about 6.72, 11.49, 16.04, 20.27, 21.77 and 24.10±0.2° 2θ.

In another aspect, the crystalline Form SMA1 of Siponimod is characterized by the PXRD pattern of FIG. 4.

The crystalline Form SMA1 of Siponimod is a complex of Siponimod and Malic acid in 1:1 w/w. The crystalline Form SMA1 of Siponimod may exist as a co-crystal of Siponimod and Malic acid in 1:1 w/w.

In another aspect, the present application provides a crystalline Form SME1 of Siponimod, characterized by a PXRD pattern comprising the peaks at about 3.64, 15.56, 16.57, 19.44 and 22.00±0.2° 2θ.

In another aspect, the present application provides a process for the preparation of crystalline Form SME1 of Siponimod, comprising,

(d) providing a mixture of Siponimod, maleic acid and a solvent,

(e) stirring the mixture of step (a), and

(f) isolating the crystalline Form SME1 of Siponimod.

The step (a) involves providing a mixture of Siponimod, maleic acid and a suitable solvent. Siponimod may be prepared by the process described in the art or may be prepared by the process described in this application. The maleic acid is used in 1:1 mole ratio with respect to Siponimod. The solvent may be taken in the ratio of 1:10 to 1:50 w/v with respect to Siponimod. The step (b) involves stirring the mixture of step (a) for about an hour to about 50 hours at above 30° C.

The step (c) involves isolation of solid from the mixture. Isolation of the solid may be carried out by any methods known in the art or procedures described in the present application. In an embodiment, the solid is isolated by filtration. The isolated solid may be washed with a suitable solvent to obtain the crystalline Form SME1 of Siponimod.

In another embodiment, the crystalline Form SME1 of Siponimod is further characterized by a PXRD pattern comprising the peaks at about 7.29, 8.28 and 24.27±0.2° 2θ.

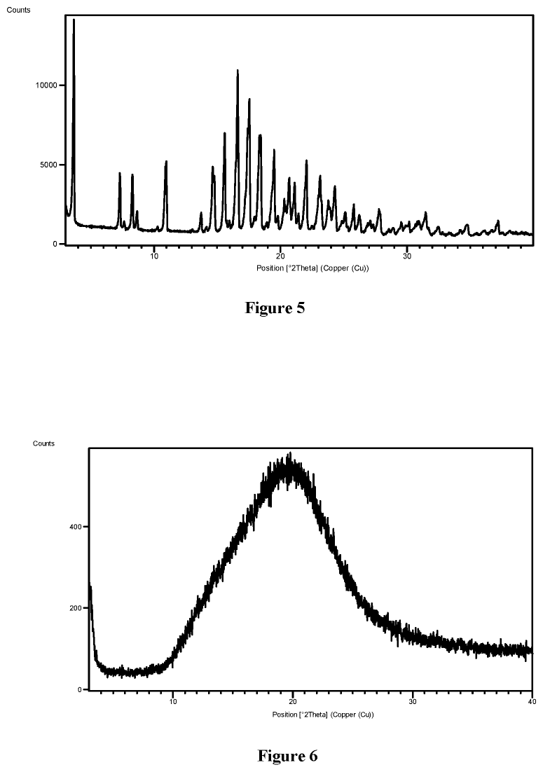

In another aspect, the crystalline Form SME1 of Siponimod is characterized by the PXRD pattern of FIG. 5.

In another aspect, the present application provides amorphous form of Siponimod.

In another aspect, the present application provides a process for preparing amorphous form of Siponimod, which comprises;
(a) providing a solution of pharmaceutically acceptable salt or a co-crystal of Siponimod in a solvent;
(b) adding a base to the solution obtained in step (a); and
(c) isolating amorphous form of Siponimod.

The Siponimod salt or the co-crystal used as the input in the process for preparation of amorphous form of Siponimod of the present application can be prepared by any process known in the art or the process described in this application.

Providing a solution of Siponimod salt in step a) includes direct use of a reaction mixture containing Siponimod salt or siponimod co-crystal that is obtained in the course of its synthesis; or dissolving Siponimod salt or siponimod co-crystal in a suitable solvent.

In one aspect the siponimod salt or the co-crystal is formed with the acid or co-former selected from the group comprising fumaric acid, maleic acid, malic acid, tartaric acid, oxalic acid, acetic acid and hydrochloric acid.

Suitable solvents which can be used for dissolving Siponimod salt or co-crystal include but are not limited to ethyl acetate, dichloromethane, water and the like; and any mixtures of two or more thereof.

After dissolution in step (a), the obtained solution may optionally be filtered to remove any insoluble particles. Suitable techniques to remove insoluble particles are filtration, centrifugation, decantation, and any other known techniques in the art. The solution can be filtered by passing through paper, glass fiber, or other membrane material, or a clarifying agent such as Celite. Depending upon the equipment used and the concentration and temperature of the solution, the filtration apparatus may need to be preheated to avoid premature precipitation of solid.

Step (b) involves adding a suitable base to the solution obtained in step (a). The base is selected form the group comprising lithium hydroxide, sodium hydroxide, sodium carbonate, sodium bicarbonate and ammonia.

Step (c) involves isolating the amorphous form of Siponimod. The isolation of amorphous form of Siponimod may be carried out by employing any of the techniques known to a person skilled in art. Techniques for the isolation of amorphous form of Siponimod include, but not limited to: decantation, filtration by gravity or suction, centrifugation, and the like, and optionally washing with a solvent.

The resulting compound in step (c) may optionally be further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 100° C., less than about 60° C., less than about 40° C., or any other suitable temperatures; at atmospheric pressure or under a reduced pressure; as long as the Siponimod is not degraded in its quality. The drying can be carried out for any desired times until the required product quality is achieved. Suitable time for drying can vary from few minutes to several hours for example from about 30 minutes to about 24 or more hours.

In another aspect, the present application provides a process for preparing amorphous form of Siponimod, which comprises;
(a) providing a solution of Siponimod in a solvent or a mixture of solvents;
(b) removing the solvent from the solution of Siponimod obtained in step a); and
(c) recovering amorphous form of Siponimod.

Siponimod used as the input in the process for preparation of amorphous form of siponimod of the present application can be prepared by any process known in the art or the process described in this application.

Providing a solution of Siponimod in step a) includes direct use of a reaction mixture containing Siponimod that is obtained in the course of its synthesis; or dissolving Siponimod in a suitable solvent.

Any physical form of Siponimod may be utilized for providing the solution of step a).

Suitable solvents which can be used for dissolving Siponimod include but are not limited to methanol, ethanol, isopropanol, ethyl acetate, dichloromethane and any mixtures of two or more thereof.

After dissolution in step (a), the obtained solution may optionally be filtered to remove any insoluble particles. Suitable techniques to remove insoluble particles are filtration, centrifugation, decantation, and any other known techniques in the art. The solution can be filtered by passing through paper, glass fiber, or other membrane material, or a clarifying agent such as Celite. Depending upon the equipment used and the concentration and temperature of the solution, the filtration apparatus may need to be preheated to avoid premature precipitation of solid.

Step (b) involves removal of the solvent form the solution obtained in step (a). Suitable techniques which can be used for the removal of solvent include but not limited to evaporation, flash evaporation, simple evaporation, rotational drying, spray drying, agitated thin-film drying, Rotary vacuum paddle dryer, agitated nutsche filter drying, pressure nutsche filter drying, freeze-drying or any other suitable technique known in the art. The drying may be carried at normal pressure or under reduced pressure.

Step (c) involves recovering an amorphous form of Siponimod. The said recovery can be done by using the processes known in the art.

In an embodiment, the isolation of amorphous form of Siponimod may be carried out by employing any of the techniques known to a person skilled in art. Techniques for the isolation of amorphous form of Siponimod include, but not limited to: decantation, filtration by gravity or suction, centrifugation, and the like, and optionally washing with a solvent.

The resulting compound in step (c) may optionally be further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 100° C., less than about 60° C., less than about 40° C., or any other suitable temperatures; at atmospheric pressure or under a reduced pressure; as long as the Siponimod is not degraded in its quality. The drying can be carried out for any desired times until the required product quality is achieved. Suitable time for drying can vary from few minutes to several hours for example from about 30 minutes to about 24 or more hours.

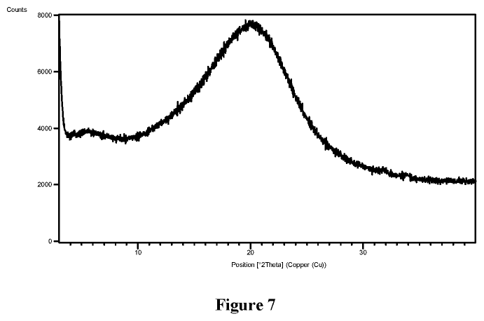

In another aspect, the present application provides amorphous form of Siponimod characterized by powder X-ray diffraction (PXRD) substantially as illustrated in FIG. 6.

In another aspect, the present application provides stable amorphous Siponimod. The amorphous Siponimod of the present application is stable for at least one year. The amorphous Siponimod prepared by the process of the present application is kept in a freezer at about 5° C. After one year the solid was analyzed using PXRD. The solid remains in amorphous form and the PXRD pattern is shown in FIG. 7.

In another aspect, the present application provides pharmaceutical composition comprising amorphous Siponimod and one or more pharmaceutically acceptable excipients.

In another aspect, the present application provides use of any of crystalline forms of Siponimod of the present invention in the purification of Siponimod or its salts and in the preparation of other crystalline forms of Siponimod.

In another aspect, the present application provides pharmaceutical composition comprising any of crystalline forms of Siponimod described in this application and one or more pharmaceutically acceptable excipients.

In another aspect, the present application provides a method of treating multiple sclerosis, comprising administering to a subject in need thereof an effective amount of any one of crystalline forms of Siponimod of the present application, or a pharmaceutical composition comprising any of crystalline forms of Siponimod of the present invention.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The following definitions are used in connection with the present invention unless the context indicates otherwise. The term “amorphous” refers to a solid lacking any long-range translational orientation symmetry that characterizes crystalline structures although; it may have short range molecular order similar to a crystalline solid.

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

All percentages and ratios used herein are by weight of the total composition and all measurements made are at about 25° C. and about atmospheric pressure, unless otherwise designated. All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example-1: Preparation of Crystalline Siponimod Form S

Glycerin (20 mL) was added to a crystallization vessel and heated to 75° C. Siponimod hemifumarate (500 mg) was added to the hot glycerin and stirred for 5 hours at 75° C. Glycerin (10 mL) was added to the mixture and cooled to 65° C. and methanol (25 mL) was added and the mixture was stirred for 2 hours at 65° C. to get clear solution. The solution was cooled to 0° C. and stirred for 10 hours. The mixture was left two days for slow evaporation at 27° C. The suspension was filtered under vacuum and sucked to dry at 27° C. PXRD as shown in FIG. 1.

### Example-2: Preparation of Crystalline Siponimod Form S

Glycerin (225 mL) was added to a crystallization vessel and heated to 85° C. Siponimod hemifumarate (5 g) was added to the hot glycerin and stirred for 1 hour at 85° C. The mixture was cooled to 65° C. and methanol (150 mL) was added and the mixture was stirred for 2 hours at 65° C. Glycerin (15 mL) and methanol (10 mL) were added to the mixture and stirred for 1 hour at 65° C. to get clear solution. The solution was cooled to 30° C. and transferred to another crystallization vessel. The mixture was left four days for slow evaporation at 27° C. The suspension was filtered under vacuum and the wet material was washed with MTBE (150 mL) and sucked to dry at 27° C. The wet material was washed again with MTBE (150 mL) and sucked to dry at 27° C. The material was dried in VTD at 40° C.

PXRD matches with FIG. 1.

### Example-3: Preparation of Crystalline Siponimod Form S1

Water (10 mL) and crystalline Siponimod Form S (0.5 g) were charged into a glass vial and the mixture was stirred for 3 hours at 25° C. The suspension was filtered under vacuum. PXRD as shown in FIG. 2.

### Example-4: Preparation of Crystalline Siponimod Form S2

0.4 g of the wet solid of crystalline Siponimod Form S1 prepared in example-3 was kept on a Petri dish and the solid was heated at 45° C. for 2 hours.
PXRD as shown in FIG. 3.

### Example-5: Preparation of Crystalline Siponimod Form S

Siponimod fumaric acid co-crystal form A (30 g) was dissolved in a mixture of Glycerol (1000 mL) and Methanol (680 mL). The mixture was heated to 60° C. The resulting clear solution was then filtered under vacuum to make it particle free. The obtained particle free filtrate was left in fume-hood for slow solvent evaporation. After 42 hours solid material was filtered and dried in vacuum tray dryer for about 15 hours at 25° C. Characterized the material using PXRD. PXRD as shown in FIG. 1.

### Example-6: Preparation of Crystalline Siponimod Form S1

Water (80 mL) and crystalline Siponimod Form S (3.31 g) were charged into a crystallization vessel and the mixture was stirred for 2 hours at 25° C. The suspension was filtered under vacuum and suck dried for 10 minutes. Characterized the material using PXRD. PXRD is shown in FIG. 2.

### Example-7: Preparation of Crystalline Siponimod Form S2

Siponimod fumaric acid co-crystal form A (2 g), Sodium bicarbonate (0.4 g), water (80 mL) were charged into a glass bottle. The resulting slurry was then stirred at room temperature for 16 hours and then filtered under vacuum to yield 0.68 g of crystalline Siponimod Form S2. PXRD as shown in FIG. 3.

### Example-8: Preparation of Crystalline Siponimod Form S2

Siponimod fumaric acid co-crystal form A (5 g), Sodium bicarbonate (1 g), demineralized water (200 mL) were charged into 500 mL RBF. The resulting slurry was then stirred at 40° C. for 15 hours and then filtered under Nitrogen using Pressure Nutch Filter to yield 2.1 g of crystalline Siponimod Form S2. PXRD as shown in FIG. 3.

### Example-9: Preparation of Crystalline Siponimod Form S2

Siponimod fumaric acid co-crystal form A (5 g), Sodium bicarbonate (1 g), demineralized water (200 mL) were charged into 500 mL RBF. The resulting slurry was then stirred at 40° C. for 1 hour. The slurry was cooled to 25° c. and dilute acetic acid (2.5 mL of 100% acetic acid in 8 mL of water) was added. Added additional 50 mL water and maintained the slurry for 30 minutes. Filtered the obtained solid material under vacuum, washed with 25 mL water and dried the obtained solid material in Air tray dryer at 30° C. for 8 hours yield 3.4 g of crystalline Siponimod Form S2. PXRD as shown in FIG. 3.

### Example-10: Preparation of Crystalline Siponimod Form S2

Siponimod fumaric acid co-crystal form A (5 g), sodium bicarbonate (0.9 g), water (220 mL) were charged into 500 mL RBF. The resulting mixture was then stirred at 25° C. for 50 minutes. Dilute acetic acid (0.33 gram of 100% acetic acid in 20 mL water) was added and maintained the slurry for about 14 hours at 25° C. Filtered the obtained solid material under vacuum and washed the wet cake with 50 mL of water. The obtained solid material was dried in Air tray dryer at 35° C. for 3.5 hours to yield 4.2 g of crystalline Siponimod Form S2. PXRD as shown in FIG. 3.

### Example-10: Preparation of Crystalline Siponimod Form S2

Siponimod form A (15 g) and water (300 mL) charged into 500 mL RBF and the mixture was stirred for 15 hours at 28° C. The solid material was filtered using Pressure Nutsche Filter and dried the solid material in Air Tray dryer at 45° C. for 1 hour to yield 11.8 g of crystalline Siponimod Form S2. PXRD as shown in FIG. 3.

### Example-11: Preparation of Crystalline Form SMA1 of Siponimod

Siponimod crystalline Form S2 (415 mg), D,L-Malic acid (106 mg) and acetonitrile (6 mL) were charged into a crystallization vial. The mixture was stirred for 44 hours at 30° C. The slurry was filtered under vacuum and the solid material was characterized using PXRD. PXRD as shown in FIG. 4.

### Example-12: Preparation of Crystalline Form SME1 of Siponimod

Siponimod crystalline Form S2 (415 mg), Maleic acid (92 mg) and acetonitrile (6 mL) were charged into a crystallization vial. The mixture was stirred for 44 hours at 30° C. The slurry was filtered under vacuum and the solid material was characterized using PXRD. PXRD as shown in FIG. 5.

### Example-13: Preparation of Amorphous Form of Siponimod

Siponimod hemifumarate (88 g), ethylacetate (880 mL) and water (880 mL) were charged into a round bottom flask. The mixture was stirred for 10 minutes at 28° C. and pH of the mixture was adjusted to 6.5 using aqueous sodium bicarbonate. The resulted mixture was stirred for 20 minutes and layers separated. The aqueous layer was extracted with ethylacetate (2×440 mL). Organic layers were combined and washed with water (520 mL). The organic layer was dried over sodium sulphate and concentrated under vacuum at 50° C. Dichloromethane (520 mL) was added to the residue and stirred for 10 minutes. The DCM layer was concentrated under vacuum at 40° C. to yield 57.2 g of off-white foamy solid. Purity: 98.75%. PXRD as shown in FIG. 6.

### Example-14: Preparation of Amorphous Form of Siponimod

Siponimod (50 g) and Dichloromethane (400 mL) were charged into round bottom flask and stirred for 10 minutes. The clear solution was filtered through a celite bed and the bed was washed with Dichloromethane (50 mL). The filtrate was concentrated under vacuum at 40° C. to yield 43.5 g of off-white solid. Purity: 98.85%. PXRD as shown in FIG. 6.

### Example-15: Stability of Amorphous Form of Siponimod

The amorphous form of siponimod prepared in example-3 was kept in an amber colored glass bottle at 2-8° C. for one year and the solid was analyzed using PXRD. PXRD as shown in FIG. 7.

1. A Crystalline Form S2 of Siponimod characterized by a PXRD pattern comprising the peaks at about 12.03, 17.68 and 20.09±0.2° 2θ.

2. The crystalline Form S2 of Siponimod of claim 1 is further characterized by a PXRD pattern comprising the peaks at about 4.03 and 8.02±0.2° 2θ.

3. The crystalline Form S2 of Siponimod of claim 1 is characterized by the PXRD pattern of FIG. 3.

4. A process for preparation of crystalline Form S2 of Siponimod of claim 1, comprising,
(a) providing a mixture of Siponimod fumaric acid co-crystal, water and a base,
(b) stirring the mixture of step (a),
(c) optionally, adding the mixture with a suitable acid, and
(d) isolating the crystalline Form S2 of Siponimod.

5. The process according to claim 4, wherein the base used in step (a) is sodium bicarbonate.

6. The process according to claim 4, wherein the Siponimod fumaric acid co-crystal used in step (a) is Form A of the co-crystal.

7. The process according to claim 4, wherein the acid used in step (c) is acetic acid.

8. A process for preparation of crystalline Form S2 of Siponimod of claim 1, comprising,
(a) providing a mixture of crystalline form of Siponimod and water,
(b) stirring the mixture of step (a), and
(c) isolating the crystalline Form S2 of Siponimod.

9. A process for preparation of crystalline Form S2 of Siponimod of claim 1, comprising drying the crystalline Form S1 of Siponimod at about 25° C. to about 60° C.

10. Crystalline Form S of Siponimod, characterized by a PXRD pattern comprising the peaks at about 6.95, 10.44, 12.12, 12.30, 17.09 and 22.11±0.2° 2θ.

11. The crystalline Form S of Siponimod of claim 10 is characterized by the PXRD pattern of FIG. 1.

12. A process for preparation of crystalline Form S of Siponimod of claim 10, comprising,
(a) providing a mixture of Siponimod fumaric acid co-crystal and glycerin,
(b) adding an alcohol solvent to the mixture of step (a), and
(c) isolating the crystalline Form S of Siponimod.

13. The process according to claim 12, wherein the Siponimod fumaric acid co-crystal used in step (a) is Form A of the co-crystal.

14. The process according to claim 12, wherein the alcohol solvent used in step (b) is methanol.

15. Crystalline Form S1 of Siponimod, characterized by a PXRD pattern comprising the peaks at about 7.18, 10.76, 12.0, 20.08 and 21.62±0.2° 2θ.

16. The crystalline Form S1 of Siponimod of claim 15 is characterized by the PXRD pattern of FIG. 2.

17. A process for preparation of crystalline Form S1 of Siponimod of claim 15, comprising,
(a) providing a mixture of crystalline Form S of Siponimod and water,
(b) stirring the mixture of step (a), and
(c) isolating the crystalline Form S1 of Siponimod.

18. Crystalline Form SMA1 of Siponimod characterized by a PXRD pattern comprising the peak at about 17.85±0.2° 2θ.

19. The crystalline Form SMA1 of Siponimod of claim 18 is characterized by the PXRD pattern of FIG. 4.

20. A process for preparation of crystalline Form SMA1 of Siponimod of claim 18, comprising,
(a) providing a mixture of Siponimod, malic acid and a solvent,
(b) stirring the mixture of step (a), and
(c) isolating the crystalline Form SMA1 of Siponimod.

21. The process according to claim 20, wherein the malic acid used in step (a) is D,L-malic acid.

22. An amorphous form of Siponimod.

23. The amorphous form of Siponimod of claim 22 is characterized by the PXRD pattern of FIG. 7.

24. A process for preparation of amorphous form of Siponimod of claim 22, which comprises;
(a) providing a solution of pharmaceutically acceptable salt or a co-crystal of Siponimod in a solvent;
(b) adding a base to the solution obtained in step (a); and
(c) isolating amorphous form of Siponimod.

25. A process for preparing amorphous form of Siponimod of claim 22, comprising,
(a) providing a solution of Siponimod in a solvent or a mixture of solvents;
(b) removing solvent from the solution of Siponimod obtained in step a); and
(c) recovering amorphous form of Siponimod.

26. A pharmaceutical composition comprising crystalline Form S2 of Siponimod, characterized by a PXRD pattern comprising the peaks at about 12.03, 17.68 and 20.09±0.2° 2θ, of claim 1, and one or more pharmaceutically acceptable excipients.

27. A pharmaceutical composition comprising crystalline Form S1 of Siponimod characterized by a PXRD pattern comprising the peaks at about 7.18, 10.76, 12.0, 20.08 and 21.62±0.2° 2θ of claim 15, and one or more pharmaceutically acceptable excipients.

28. A pharmaceutical composition comprising crystalline Form S of Siponimod characterized by a PXRD pattern comprising the peaks at about 6.95, 10.44, 12.12, 12.30, 17.09 and 22.11±0.2° 2θ of claim 10, and one or more pharmaceutically acceptable excipients.

29. A pharmaceutical composition comprising crystalline Form SMA1 of Siponimod characterized by a PXRD pattern comprising the peak at about 17.85±0.2° 2θ of claim 18, and one or more pharmaceutically acceptable excipients.

30. A pharmaceutical composition comprising amorphous form of Siponimod of claim 22, and one or more pharmaceutically acceptable excipients.
